(19) United States
(12) Patent Application Publication
(10) Pub. No.: US 2023/0248602 A1
(43) Pub. Date: Aug. 10, 2023

(54) BIMODAL EXOSUIT

(71) Applicant: Vanderbilt University, Nashville, TN (US)

(72) Inventors: Karl ZELIK, Nashville, TN (US); Jaime FINE, Clarksville, TN (US); Anna WOLFE, Nashville, TN (US)

(21) Appl. No.: 18/010,756

(22) PCT Filed: Jun. 16, 2021
(86) PCT No.: PCT/US2021/037579

§ 371 Date: Dec. 15, 2022

Related U.S. Application Data
(60) Provisional application No. 63/039,869, filed on Jun. 16, 2020.

(51) Int. Cl. A61H 3/00; A61H 1/02

(52) U.S. Cl. CPC A61H 3/00; A61H 1/0244; A61H 2201/163; A61H 2201/1253; A61H 2201/1616; A61H 2201/1626; A61H 2201/1652

(57) ABSTRACT

Wearable assistance devices and methods of using the same are disclosed. Embodiments relate to bimodal wearable assistance devices that provide assistance in one mode, and no or reduced assistance in a second mode, for reducing muscle stress, fatigue, injury and/or pain in the lower back or other body segments, and enabling comfortable, free or increased range of motion. Other embodiments relate to interfaces for wearable assistance devices that can loosen/tighten.

### CROSS REFERENCE TO RELATED APPLICATION(S)

This application claims priority to U.S. Provisional Pat. Application Serial No. 63/039,869, filed on Jun. 16, 2020, which is hereby incorporated herein by reference in its entirety.

### GOVERNMENT SPONSORSHIP

This invention was made with government support under grant R01 EB028105 awarded by the NIH. The government has certain rights in the invention.

### FIELD OF THE INVENTION

Embodiments are in the field of wearable assistance devices such as exosuits/exoskeletons. More particularly, embodiments disclosed herein relate to bimodal wearable assistance devices for reducing muscle stress, fatigue, injury and pain in the lower back or other body segments.

### BACKGROUND OF THE INVENTION

In recent years, there has been rapid growth in the development of occupational exoskeletons and exosuits, and these technologies are being adopted for various industrial, clinical, recreational and military applications. Despite the promising trajectory of these devices, a number of factors have limited their rate of adoption. Critical among these limiting factors are comfort, form-factor, and movement interference. Users are unlikely to adopt a wearable device if it is uncomfortable or if it interferes with their movement range-of-motion or daily tasks (e.g., sitting down). Exosuits/exoskeletons must be able to provide assistance to users when it is needed, but to stay out of the way and remain comfortable when assistance is not needed.

Thus, it is desirable to provide a wearable assistance device and method of using a wearable assistance device that are able to overcome the above-described disadvantages.

Advantages of the present invention will become more fully apparent from the detailed description of the invention hereinbelow.

### SUMMARY OF THE INVENTION

Embodiments are directed to a wearable assistance device configured to be worn by a user. The device includes: an upper body interface; a lower body interface; one or more elastic members, each of the elastic members mechanically coupling the upper body interface to the lower body interface, and extending from the upper body interface to the lower body interface along a first route traversing a body segment of the user, to form an engaged mode, to apply an assistive force to and/or assistive moment about the body segment of the user; and an engagement/disengagement system mechanically connected to the one or more elastic members that allows the one or more elastic members to move, shift or rotate from along the first route traversing the body segment in the engaged mode to a second route different than the first route, to form a disengaged mode, that slackens the one or more elastic members and/or lessens or prevents the assistive force applied to the body segment.

Embodiments are also directed to a wearable assistance device configured to be worn by a user. The device includes an interface and an elastic member mechanically coupled to the interface via a connection system via a first force when in an engaged mode, and the elastic member is mechanically coupled to the interface via the connection system via a second force less than the first force when in a disengaged mode. The interface is configured to be worn on a body part of the user via a first tension when in the engaged mode, and via a second tension less than the first tension when in the disengaged mode.

Additional embodiments and additional features of embodiments for the wearable assistance device and method of using a wearable assistance device are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It is understood, however, that the inventive concepts disclosed herein are not limited to the precise arrangements and instrumentalities shown in the figures. The detailed description will refer to the following drawings in which like reference numerals, where present, refer to like items.

FIGS. 1A-1C are drawings illustrating an exosuit worn by a user and having an engagement/disengagement system comprising cording/cables, in an engaged mode;

FIGS. 2A-2E are drawings illustrating the exosuit worn by the user as shown in FIGS. 1A-1C and having the engagement/disengagement system comprising cording/cables, in a disengaged mode;

FIG. 3 is a drawing illustrating an exosuit worn by a user and having an engagement/disengagement system comprising hinges, in a disengaged mode;

FIG. 4 is a drawing illustrating the exosuit worn by the user as shown in FIG. 3 and having the engagement/disengagement system comprising hinges, in an engaged mode;

FIG. 5 is a drawing illustrating an exosuit worn by a user and having an engagement/disengagement system comprising slidable hubs, in a disengaged mode;

FIG. 6 is a drawing illustrating the exosuit worn by the user as shown in FIG. 5 and having the engagement/disengagement system comprising slidable hubs, in an engaged mode;

FIG. 7 is a drawing illustrating an exosuit worn by a user and having an engagement/disengagement system comprising extensible moment arms, in a disengaged mode;

FIG. 8 is a drawing illustrating the exosuit worn by the user as shown in FIG. 7 and having the engagement/disengagement system comprising extensible moment arms, in an engaged mode;

FIG. 9A is a drawing illustrating an exosuit worn by a user and having an engagement/disengagement system comprising a zipper, in a disengaged mode (dashed lines) and an engaged mode (solid lines). FIG. 9B is an enlarged drawing illustrating the engagement/disengagement system comprising the zipper as shown in FIG. 9A, in the engaged mode. FIG. 9C is an enlarged drawing illustrating the engagement/disengagement system comprising the zipper as shown in FIG. 9A, in the disengaged mode;

FIGS. 10A-10B are drawings illustrating a cordlock mechanism which can be part of an engagement/disengagement system of an exosuit;

FIGS. 11A-11B are drawings illustrating hubs which can be part of an engagement/disengagement system of an exosuit;

FIGS. 12A-12C are drawings illustrating loosening/tightening of a lower body interface (of an exosuit worn by a user) comprising a (e.g., leg) sleeve, in an engaged (tight / form-fitting interface) mode;

FIGS. 13A-13C are drawings illustrating loosening/tightening of the leg sleeve shown in FIGS. 12A-12C, in a disengaged (loose interface) mode;

FIGS. 14A-14C are drawings illustrating an exosuit worn by a user (and which is similar to the embodiment in FIGS. 13A-13C), in a disengaged mode, but the leg sleeves do not tighten/loosen in this embodiment;

FIGS. 15A-15C are drawings illustrating the exosuit shown in FIGS. 14A-14C, in an engaged mode;

FIGS. 16A-16B are illustrations of an exosuit worn by a user and having an engagement/disengagement system comprising extensible moment arms which are non-extended, in a disengaged mode;

FIGS. 17A-17B are illustrations of the engagement/disengagement system shown in FIGS. 16A-16B comprising extensible moment arms which are extended, in an engaged mode;

FIG. 18 is a drawing illustrating an exosuit worn by a user and having two different elastic members on top of each other, which can be used for multi-modal functionality or non-linear stiffness assistance (e.g., by incorporating elastic members with different stiffness or slack lengths);

FIG. 19 is a drawing illustrating loosening/tightening of a lower body interface (of an exosuit worn by a user) comprising a (e.g., leg) sleeve where a small portion of the leg sleeve remains tight around the body segment;

FIG. 20A is a drawing illustrating an exosuit worn by a user and having an engagement/disengagement system having a manual or motorized pull to disengage, in an engaged mode. FIG. 20B is a drawing illustrating the engagement/disengagement system shown in FIG. 20A and having retractable springs or motors to engage/disengage, in a disengaged mode;

FIGS. 21A-21C are drawings illustrating an exosuit worn by a user and having three modes: disengaged mode, engaged mode 1, and engaged mode 2, respectively;

FIGS. 22A-22B are drawings illustrating loosening/tightening of an upper body interface (of an exosuit worn by a user) comprising, for example, a vest, in a disengaged (loose interface) mode;

FIGS. 23A-23B are drawings illustrating loosening/tightening of the vest shown in FIGS. 22A-22B, in an engaged (tight / form-fitting interface) mode;

FIG. 24A is a drawing illustrating an exosuit (i.e., an arm exosuit) worn by a user and assisting a body segment other than the back, in an engaged mode. FIG. 24B is a drawing illustrating the arm exosuit shown in FIG. 24A, in a disengaged mode;

FIG. 25 is a drawing illustrating a back view (in an engaged mode) and a side view (where the connector would be located in the upper right portion of the channel, in a disengaged mode) of a thigh interface comprising a channel (or groove) configured to allow a connector (attachment point as shown in the figure) to slide therein, wherein the connector is coupled to a spring (or other elastic member);

FIGS. 26A-26B are drawings illustrating perspective views of a thigh sleeve interface (in engaged (tight) mode and disengaged (loose) mode, respectively) comprising a channel (or groove) in an outer layer thereof, similar to the channel (or groove) shown in FIG. 25; and

FIG. 27 is a drawing illustrating a perspective view of a spring (elastic element) attached to webbing (or other elastic member) and a thigh sleeve, in which stretching the spring would also tighten the thigh sleeve.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical wearable assistance device or typical method of using a wearable assistance device. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

For purposes of this disclosure, the phrase “body segment” may include a body part such as a back, lumbar spine, hip, neck, etc., or a body joint such as an ankle, knee, elbow, wrist, etc., and thus, may all be used interchangeably. Also, the phrase “body segment” may include multiple body parts or body joints.

For purposes of this disclosure, the phrase “wearable assistance device” may be an exosuit, exoskeleton, or other device that provides assistive force to and/or assistive moment about a body segment of a user, and may be passive, quasi-passive, or powered.

For purposes of this disclosure, the terms “channel”, “track”, and “groove” may all be used interchangeably.

For purposes of this disclosure, the phrase “elastic member” may be any member that has an amount of elasticity associated with it and which can take the form of, for example, a spring, cable, string, strap, cord, webbing, rope, band, gas-spring, pneumatic, carbon fiber, fiber glass, etc., and may be coiled or non-coiled.

For purposes of this disclosure, the phrases “upper body interface” and “lower body interface” refer to body interfaces that can be positioned anywhere on the user’s body, with the upper body interface placed higher relative to the lower body interface, assuming the user is in a standing/vertical position.

For purposes of this disclosure, the phrase “clutch mechanism” or “clutch” may include any device that engages and disengages mechanical elements (e.g., elastic members or portions thereof) that bear or transmit force or mechanical power. A clutch mechanism may be unpowered such that it engages and disengages based on manual input or movement from the user, and may include one or more springs that bias the clutch towards a nominal mode (e.g., on or off). Alternatively a clutch mechanism may be powered such that a motor or other actuator with its own power supply is used to control engagement and disengagement. Additionally a motor or other actuator may be used to control the position of clutch engagement relative to one or more mechanical elements (e.g., elastic members), or to control the set point of an elastic member relative to the position of clutch engagement, thereby adjusting or setting tension of, for example, elastic member(s). The clutch mechanism may be used in combination with additional motors or other actuators that provide tensile force or perform work along (parallel with) the elastic members, or provide force transverse or perpendicular to the elastic members. The engaging and disengaging by the clutch mechanism of a mechanical element may be achieved by any form of clutch or brake or mechanism providing a similar function, for example, a ratchet, dog clutch, cam clutch, friction clutch, overrunning clutch, disc brake, drum brake, latch, buckle, variable-ratio gear, or other resecurable fastening device.

For purposes of this disclosure, the phrase “leaning” is interchangeable with the terms “hinging”, “flexing”, “extending”, “bending”, “stooping”, and/or any combination of these movements or postures.

For purposes of this disclosure, the phrase “cable” is interchangeable with the terms “string”, “strap”, “cord/cording”, “webbing”, “rope”, “band”, and/or any combination thereof.

Embodiments described herein relate to a bimodal wearable assistance device for the back or other body segment of a user that provides assistance in one mode (i.e., an engaged mode), and no or reduced assistance in a second mode (i.e., a disengaged mode) for reducing lower back (or other body segment) muscle stress, fatigue, injury and/or pain, and enabling free or increased range of motion. This new concept achieves this bimodal design without requiring a clutch, although a clutch may optionally be employed. These embodiments preferably use kinematics/geometry to differentiate the behavior of the device in engaged mode and disengaged modes, rather than a mechanical clutch to toggle assistance on/off or a powered actuator to directly control force in the elastic members, though these elements may also be included in some embodiments.

The bimodal exosuit is engaged (assist/activated mode) when the elastic member(s) are aligned along a first route traversing a body segment (e.g., a back of a torso of a user, such that when the user bends forward, the elastic members stretch and/or increase their force, which offloads/reduces the force needed by the user’s back muscles). To disengage/deactivate, a mechanism or force from the user moves the elastic member(s) so they are aligned along a different route (e.g., a side of the torso of the user, such that when the user bends forward, the elastic members do not stretch and/or substantially increase their force) in a way that does not impede range of motion.

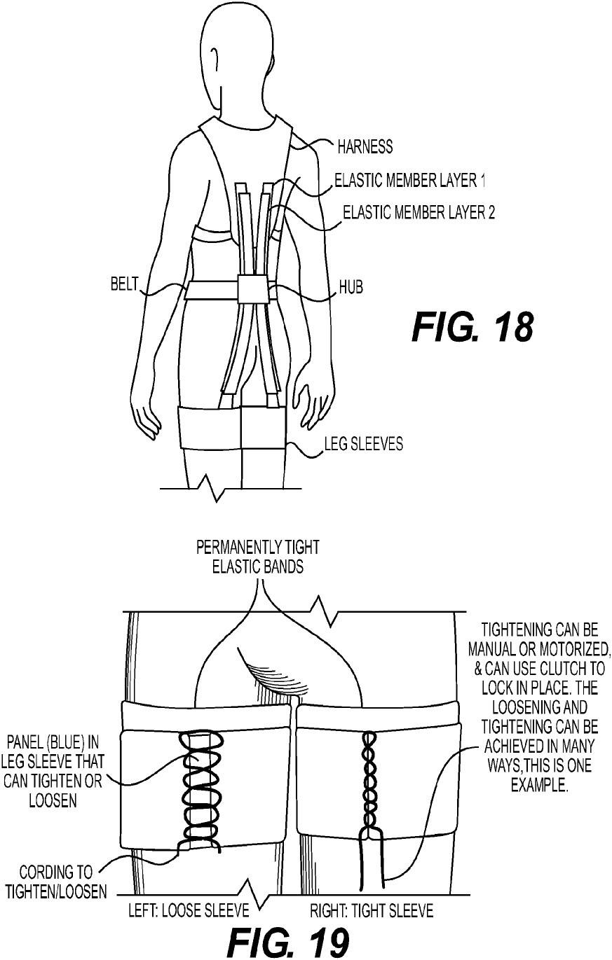

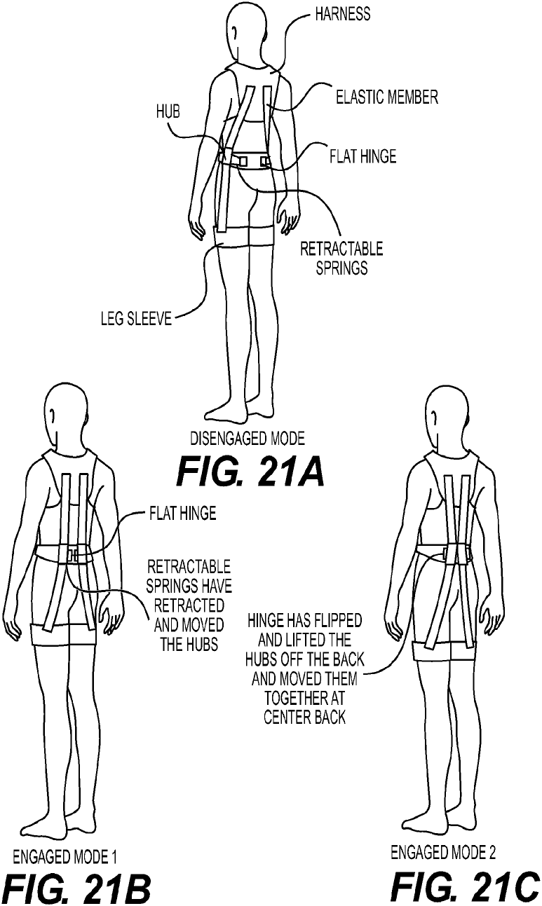

FIGS. 21A-21C are drawings illustrating a multi-modal exosuit worn by a user and having three modes: disengaged mode, engaged mode 1, and engaged mode 2, respectively. Elastic members are on the sides of the user in disengaged mode (FIG. 21A). Retractable springs move the elastic members closer to the center back for engaged mode 1 (FIG. 21B). Then a hinge lifts the elastic members off the back and moves them all the way together at center back for a mini extensible moment arm for engaged mode 2 (FIG. 21C). The exosuit may alternatively have more than two modes and is capable of switching between these modes: 1) activated/engaged mode aligns elastic elements of the exosuit behind the user to reduce back strain during lifting and bending; and 2) deactivated/disengaged mode aligns the elastic elements along the sides of the user so they can move freely and sit down comfortably. There could be more than one activated mode, for instance, if there were two or more sets of elastic members then the user could engage lower vs higher levels of assistance (e.g., with elastic members having two different levels of tension/elasticity) in the activated mode. As an example, FIG. 18 is a drawing illustrating an exosuit worn by a user and having two different elastic members on top of each other. This embodiment shows two different elastic members layered on top of each other for an exosuit. That could be used to create more than two modes (assistance levels) or to customize non-linear elastic properties. The mode switching could be driven/controlled by powered (e.g., motorized) or passive (e.g., manual or elastic) inputs.

The interface is the part of the exosuit that physically anchors/connects to the user’s body. The terms lower and upper-body interface are relevant to embodiments of the back-assist exosuit. Nominally, the lower body interface is leg or thigh sleeves, but could also be pants, shorts, soft wrap, rigid or semi-rigid shell, etc. Nominally, the upper body interface is a harness, but could also be a shirt, vest, bra, baby carrier or made from other soft, rigid, or semi-rigid components.

In the simplest sense, the lower body interface is a component that connects/anchors to each leg or thigh. Nominally, the back side of the leg sleeve connects to the elastic member(s), but the elastic member(s) could also be connected to other sides or surfaces of the lower body interface.

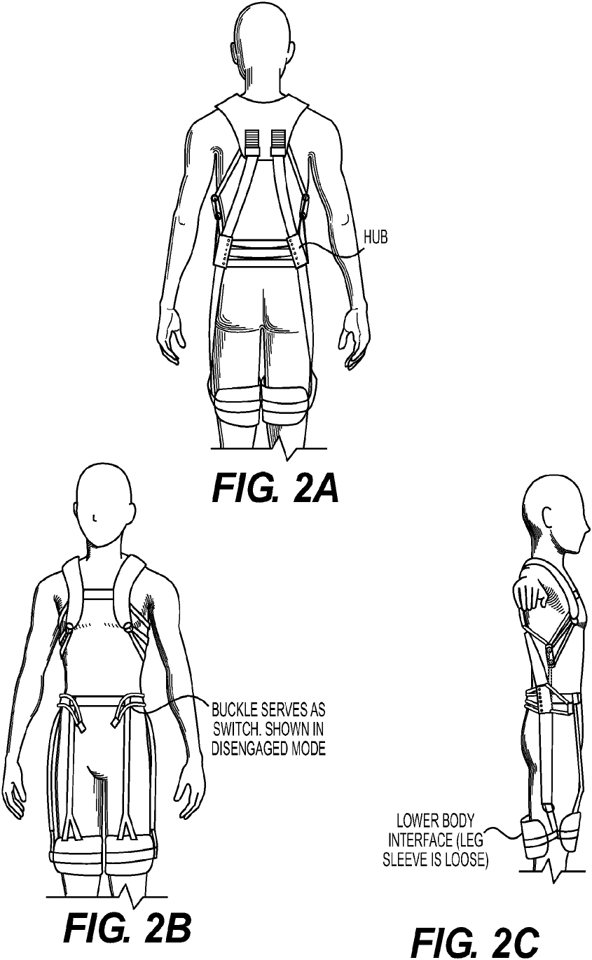

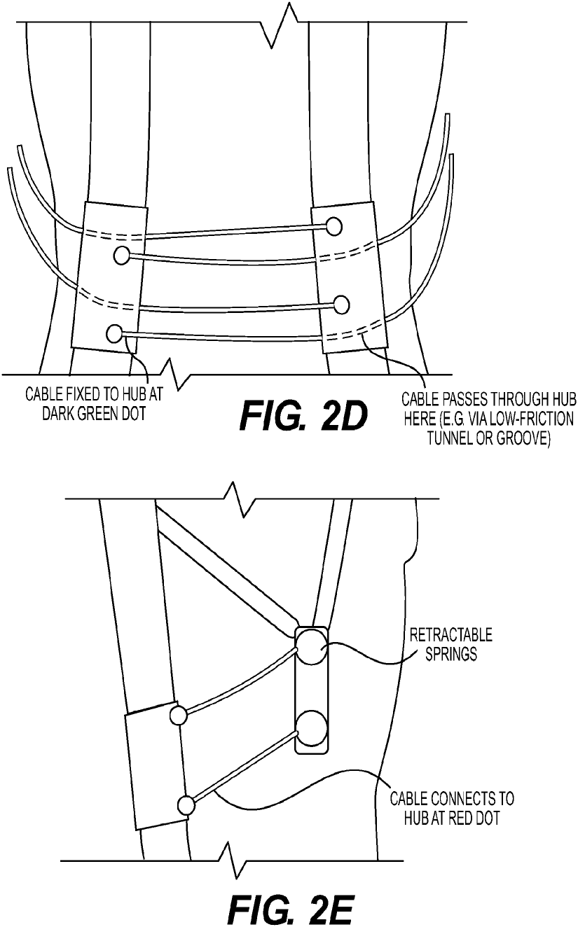

In an embodiment, the lower body interface (e.g., thigh sleeve) automatically loosens in the disengaged mode & tightens on the thigh when engaged. This dynamic loosening behavior occurs because of the gray webbing wrapped around the green textile portions of the leg sleeve, as shown in FIG. 2C. Here the gray webbing passes through a simple loop such that the circumference of the thigh sleeve can increase. The free end of the gray webbing is connected up to the elastic member(s). When the elastic members are moved to side of user, this slackens the gray webbing allowing leg sleeve to loosen.

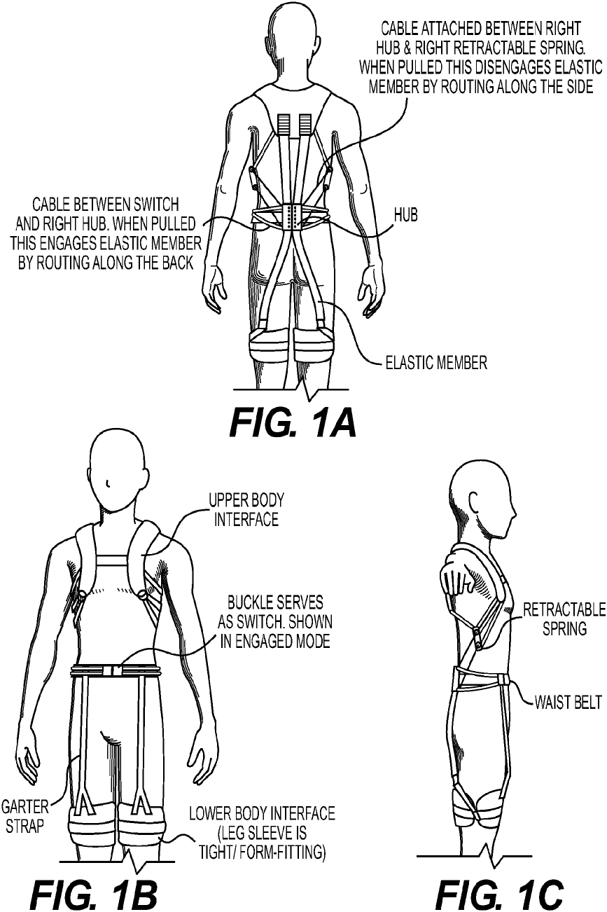

With reference to FIGS. 1A and 1C, when the elastic member(s) move to a back side of user, the gray webbing is pulled taut causing the thigh sleeve to tighten around the user’s leg (engaged mode). There can also be a hardstop added to the webbing to prevent over-tightening of leg sleeve when the elastic member(s) are loaded.

The upper body interface must anchor the elastic members to the upper body (e.g., trunk and/or shoulders). This component can have varying degrees of coverage of the trunk and can take many different forms, including but not limited to: harness, vest, jacket, shirt and backpack, etc.

Each elastic member couples the upper body interface to the lower body interface. An elastic member is also connected to the engagement/disengagement system, e.g., at the lower center of the back (see, for example, FIG. 4). The dark gray boxes are a portion of the engagement/disengagement system called the hubs (including, for example, right and left hubs).

Each elastic member could include one or more elastic elements. For instance, the image shown in FIG. 4 shows two elastic members, each of which includes a top elastic element, a bottom elastic element, and a hub that connects these two elastic elements. An elastic member or element could also be replaced with another type of material or actuator such as a damper, viscoelastic material, series elastic actuator or other motorized actuator, or fluid-power actuator.

FIGS. 1A-1C are drawings illustrating an exosuit worn by a user and having an engagement/disengagement system comprising cording, in an engaged mode. This embodiment employs an unpowered, manual bimodal exosuit and loosening/tightening leg sleeves, and is shown in engaged mode. There are two mechanisms involved in switching modes: retractable coil springs that sit at the side of the body (small gray circles and red cording) for switching to disengaged mode, and the hubs with green cording attached and routed through them for switching to engaged mode. To engage the suit, the green cording that is routed through the hub is pulled towards the front of the body and buckled into place. Cording on the right side of the body pulls the left hub towards the center back, and cording on the left side of the body pulls the right hub towards the center back. When the hubs are pulled towards the center back, they pull the elastic members towards the center back, which pulls webbing in the thigh sleeves tight.

FIGS. 2A-2E are drawings illustrating the exosuit worn by the user as shown in FIGS. 1A-1C and having the engagement/disengagement system comprising cording, in a disengaged mode. In order to disengage, when the buckle that is holding the green cording in place is unlocked, the retractable springs at the side of the body (gray circles with red cording) automatically pull the hubs apart and bring the elastic members to the side of the body. That in turn gives slack to the leg sleeves and causes them to loosen.

The elastic members are deactivated by releasing the engagement cording via a front switch. In this embodiment, the on/off switch is a simple buckle. When the buckle is released (disengaged mode), retractable springs on the sides of the body (attached via the cording at the hubs on the elastic members) will automatically pull the elastic members apart and to the side of the body. In other words, after unbuckling the deactivation of the suit is done automatically with retractable springs on each side of the body, which are attached to the hubs on the elastic members. The cording is pulled along the sides of the body and the buckle is clipped together to activate the suit in the engaged mode. In this embodiment shown, the mode-switching is triggered manually by the user, by bucking or unbuckling. However, the retractable springs and/or switch could be replaced by different types of passive mechanisms (e.g., springs, bi-stable mechanisms) or by a powered actuator unit (e.g., including a motor, battery and processor) which optionally may include a clutch, gears, or other mechanical transmission mechanisms. For powered embodiments, mode-switching could then be controlled by sensor inputs, user input, and/or an automated control algorithm on the processor.

The engagement/disengagement system pulls the elastic members together in the center of the back. In the embodiment shown in FIGS. 1A-2E, there are two hubs (rectangles) near the lower back, which are connected to cords. The cord from the right (and left) hub crosses the back and connects up to the left (and right) strap on the front side of the harness. There is also a switch mechanism located on the front of the body. When the switch is activated, the cords are pulled taut, which in turn pull the hubs and thus the elastic members into engaged mode.

FIG. 2D shows an enlarged portion of one embodiment of the engagement mechanism part of the engagement/disengagement system, in which cables connect from the switch (not depicted) around one side of the body and connect to the hub (and/or elastic member) on the other side of the body. When the free ends of the cables are pulled, the hubs and elastic members are pulled to the back of the body. FIG. 2E shows an enlarged portion of one embodiment of the disengagement mechanisms parts of the engagement/disengagement system. This uses retractable coil springs such that when the switch is disengaged (e.g., buckle on front side disconnects) then the two red (side) cables retract inside the retractable spring housing (similar to a key chain retractor) and the hubs and elastic members are pulled to the side of the wearer.

How the manual pull to engaged works: There are two pieces of cording affixed to the left hub, that are routed through the right hub and sit on the right side of the body. When this cording on the right side of the body is pulled forward, it pulls the left hub center back. This same process is applied to the right hub as well.

How the automatic disengage works: When the hubs are manually pulled together in engaged mode, the retractable springs on the sides of the body are stretched. When the clip holding the suit in engaged mode is unlocked, the stretched retractable springs automatically retract, applying force to the hubs and/or elastic members that pull them to the sides of the body.

The switch is a catch-all term for any type of mechanism, or set of mechanisms (e.g., switches, pulls, triggers, toggles, actuators) that have two or more states that control the mode of the exosuit. The switch can either receive (e.g., from the exosuit user) a force or displacement input to change from one mode to the other, or it can receive an input (e.g., sensor, manual, force, displacement) that then triggers another force or displacement to change the exosuit from one mode to the other. Examples of switch mechanisms may include: buckles/straps whereby the user would pull on the straps and clip the buckle to lock the system into engaged mode then unbuckle to disengage, or a rotational dial that a user would turn one direction to put the system into engaged mode and then rotate the opposite direction to put the system into disengaged mode, or any other mechanism by which a force or displacement could be applied manually by the user to switch modes, or by a motor or other actuator (controlled by sensors and microprocessors and powered by batteries or other sources) to switch modes. The switch may control both engagement and disengagement, although the physical action or mechanism may be different for engagement than disengagement. For instance, the user might manually pull on a strap to switch to engaged mode, but then press a small button on a cordlock mechanism to switch to disengaged mode. In addition to cords, other transmissions could also be used such as cables or straps. The switch can be passive or powered (e.g., motorized). There may be one or more switches. The bimodal function (rerouting) of elastic bands can be used with or without loosening loosening/tightening interfaces. And the loosening/tightening interfaces can be used with or without the bimodal function (rerouting) of elastic bands.

The hub can be affixed directly to the elastic members, or may slide/up down on the elastic members, potentially with its own spring-mechanism to bias the motion of the hub to one direction (i.e., biased to nominally be in engaged mode, or in disengaged mode).

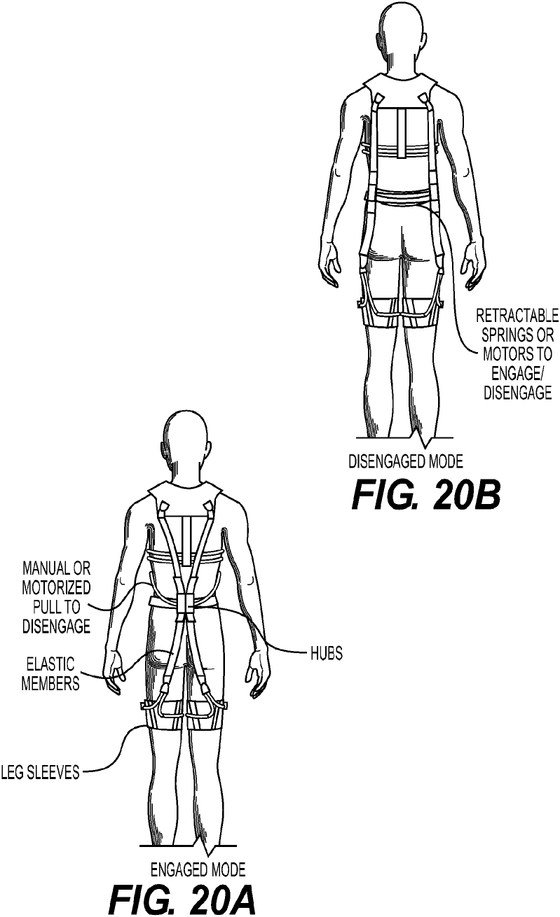

The engagement/disengagement system is also used to transition from engaged to disengaged mode. FIG. 20A is a drawing illustrating an exosuit worn by a user and having an engagement/disengagement system having a manual pull or a motor (or other powered actuator) to switch modes, in an engaged mode. FIG. 20B is a drawing illustrating the engagement/disengagement system shown in FIG. 20A and having retractable springs or motors to engage/disengage, in a disengaged mode. In this embodiment, there are retractable spring components along both sides of the body. These attach to a second set of cords which are positioned to pull each hub towards the lateral side of the users. The transition from engaged to disengaged is accomplished by deactivating the switch on the front of the body. This allows the retractable springs to automatically pull the hubs (and thus the elastic members) apart. In one embodiment, there is a retractable spring used to disengage, and a manual (or powered) switch is used to engage. In another embodiment, there is a retractable spring used to engage, and a manual (or powered) switch is used to disengage. In another embodiment, there is a manual (or powered) switch used to engage, and a manual (or powered) switch is used to disengage.

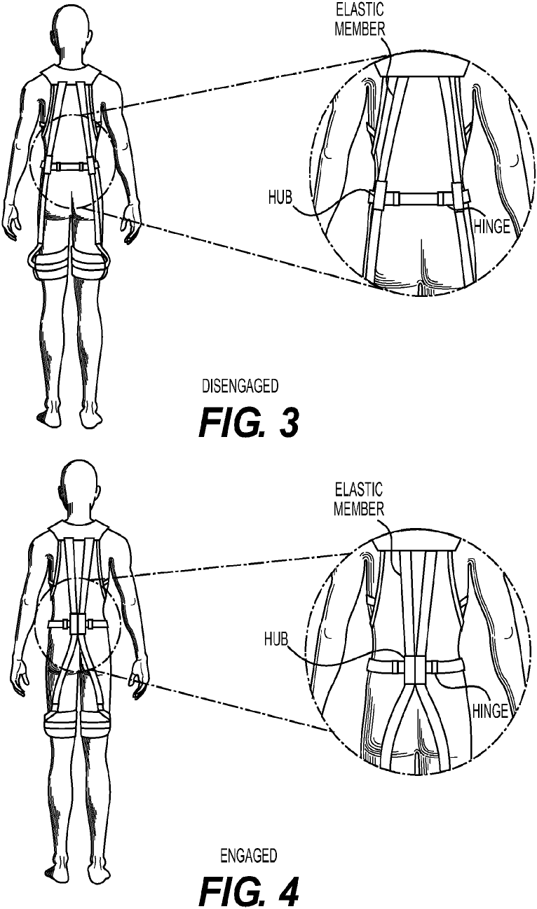

FIG. 3 and FIG. 4 show a hinged embodiment which is an example where a clutch mechanism is optional, but not necessary. That is because when the elastic members are routed along the back of the torso during engaged mode they will tend to remain in that location (since forces are applied in tension along the bands), and likewise when the elastic members are routed along the sides of the torso during disengaged mode they will also tend to remain in that location since forces in the slackened bands are small or negligible.

FIG. 3 is a drawing illustrating an exosuit worn by a user and having an engagement/disengagement system comprising hinges or axes, in a disengaged mode. When the disengaged mode is activated, the hinges/axes on the belt at the lower back are rotated away from the center of the back, moving the routes of the elastic members to be along the sides of the torso. The hinges themselves can be designed using a bi-stable mechanism (e.g., similar to in a 3-ring binder) such that the elastic members stay in engaged mode, or stay in disengaged mode, until the switch is activated to thereby change the mode. This embodiment may optionally include a clutch of the type, for example, in any of the embodiments described in this disclosure.

FIG. 4 is a drawing illustrating the exosuit worn by the user as shown in FIG. 3 and having the engagement/disengagement system comprising hinges or axes, in an engaged mode. When the engaged mode is activated, the hinges/axes on the belt at the lower back are rotated towards the center of the back, moving the routes of the elastic members to be along the back of the torso.

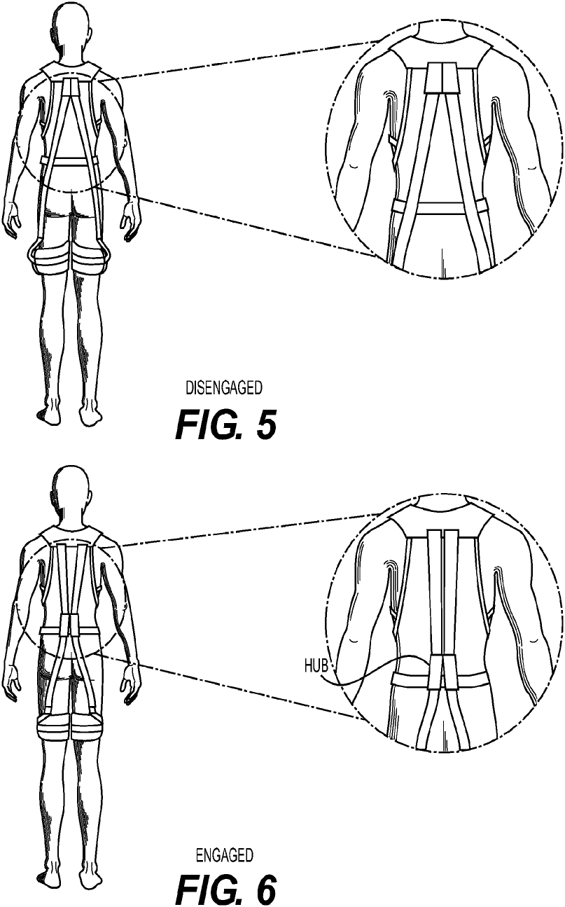

FIG. 5 is a drawing illustrating an exosuit worn by a user and having an engagement/disengagement system comprising slidable hubs (i.e., left and right hubs) that are affixed to each other, in a disengaged mode. Each slidable hub is slidably connected to a corresponding elastic member. In the disengaged mode of FIG. 5, the slidable hubs are positioned at the upper portions of the elastic members.

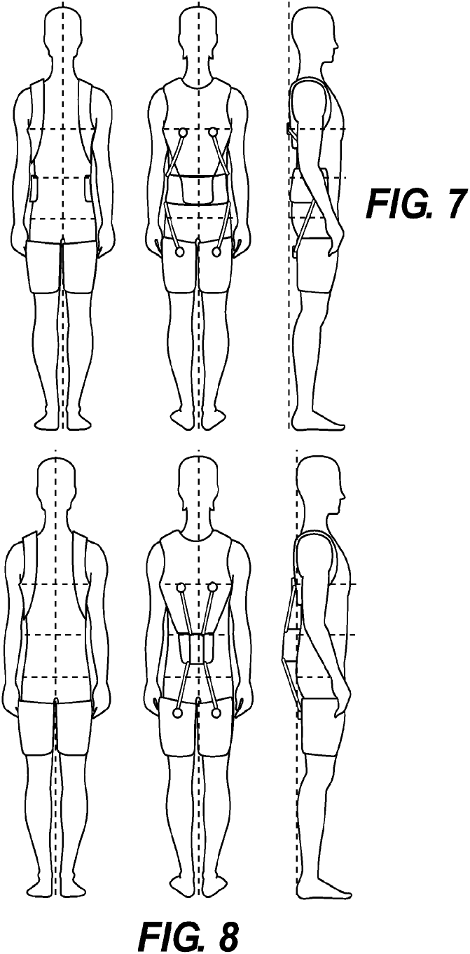

FIG. 6 is a drawing illustrating the exosuit worn by the user as shown in FIG. 5 and having the engagement/disengagement system comprising slidable hubs that are affixed to each other, in an engaged mode. In the engaged mode of FIG. 6, the slidable hubs are lowered to positions substantially mid-way between ends of the elastic members (e.g., at or near a waist or hip of the user). Because the slidable hubs are affixed to each other, the elastic members are brought together at the mid-back of the user as the slidable hubs are lowered towards the mid-back.

FIG. 7 is a drawing illustrating an exosuit worn by a user and having an engagement/disengagement system comprising extensible moment arms, in a disengaged mode. This embodiment shows how extensible moment arms could be included in the exosuit. The key addition is a flap at the lower back which includes 3 panels/flaps. This functionality could also be accomplished using other mechanisms, such as the hinges used in FIG. 3 and FIG. 4. In disengaged mode, as shown, these panels wrap around the body in a form-fitting manner. The elastic members can be attached (e.g., fixed connection, sliding connection) directly to the most lateral aspects of the outer flaps, or alternatively hubs may be included as an intermediate connection.

FIG. 8 is a drawing illustrating the exosuit worn by the user as shown in FIG. 7 and having the engagement/disengagement system comprising extensible moment arms, in an engaged mode. When engaged, the engagement/disengagement system pulls the elastic members and the outer flaps towards the middle of the back. The outer flaps can rotate about hinges or axes at the sides of the middle flap. Thus, in addition to moving towards the center of the back, the outer flaps also create a larger protrusion from the back. That increases the mechanical advantage (assistive torque) via a lever arm effectively provided by the elastic members, which has potential to increase assistance magnitude and/or increase comfort. That provides an extended moment arm which allows the wearable assistance device to provide more joint torque to the user per unit force going through each elastic member.

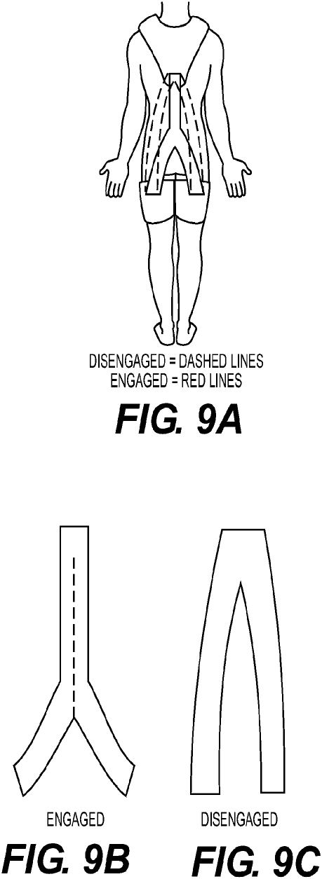

FIG. 9A is a drawing illustrating an exosuit worn by a user and having an engagement/disengagement system comprising a zipper (or other fastening mechanism), in a disengaged mode (dashed lines) and an engaged mode (solid lines). FIG. 9B is an enlarged drawing illustrating the engagement/disengagement system comprising the zipper as shown in FIG. 9A, in the engaged mode (i.e., with the zipper fastened). FIG. 9C is an enlarged drawing illustrating the engagement/disengagement system comprising the zipper as shown in FIG. 9A, in the disengaged mode (i.e., with the zipper unfastened).

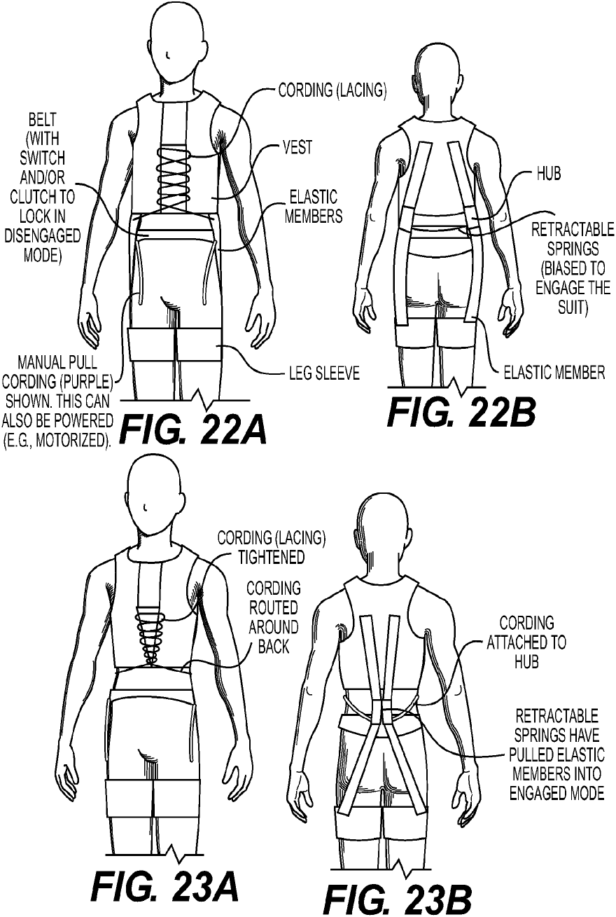

Embodiments described herein also relate to a dynamically-adaptive or mode-switching interface (such as the vest for the exosuit shown in FIGS. 22A-23B described below) which enables one or more interfaces of the exosuit to be more breathable, and worn more loosely when not engaged or providing assistance. A goal is to have interfaces that can tighten or loosen on a user’s body part (e.g., thigh, shank, upper arm, lower arm, trunk). The interface refers to the material, sleeve or other element that fits on or around a user’s body part (e.g., a shank sleeve or shoulder harness) to transmit forces from the assistive exosuit to the user. The interface may be integrated into clothing or other apparel, or may be a separate component.

In one iteration of this design, the interface (e.g., sleeve) is loose when the device is not in use (i.e., disengaged mode). For instance, the sleeve or material might fit like a pair of loose shorts. However, when the assistive force of the wearable assistance device is needed, then the interface would tighten on the body (using a passive or powered (e.g., motorized) mechanism) so that force could then we transmitted to the body from the wearable assistance device. The switch that controls loosening/tightening may be located on the interface itself, or located elsewhere on the exosuit or users body by using a transmission system (e.g., Bowden cable or other flexible conduit). In that tightened state, the interface might fit more like spandex or compression shorts.

In another iteration/embodiment, the “looser” state would be the compression short tightness. When the exosuit is bearing a load, the interface would then tighten even further. For example, the interface would become tighter as the exosuit pulling force increased. There may optionally be a mechanism in or coupled to the interface that limits the maximum tightening force (e.g., using a hardstop). Alternatively or additionally, the amount of tightening could be controlled along a continuum by an actuator (e.g., motor) and sensor, for instance, such that it could be controlled to dynamically loosen or tighten for different activities.

In one embodiment, interface tightening (and loosening) could be controlled by a manual switch or tactile sensors on the user’s body or on the exosuit that the user triggers to switch the mode. In another embodiment, an automated algorithm may use wearable sensor inputs to monitor motion or biometric data from the user, and automatically adjust interface tension (e.g., tightening on the body segment during bending motions, and loosening during static postures). The automated algorithm may be on a processor in the exosuit, or alternatively located off the user/exosuit and will transmit control signals wirelessly.

In another embodiment, the sensors, receivers or transmitters may be, in part, not worn on the user’s body. For example: proximity sensors, Bluetooth or video (with image processing or motion tracking capabilities) could be used to automatically turn the exosuit on/off as a user enters/exits a vehicle (e.g., delivery truck) or other specified area. In one exemplary embodiment, the exosuit would automatically turn off when it was more than 5 feet away from a delivery truck, since assistance is generally not needed during walking/carrying away from the truck. In another embodiment, if the exosuit was away from the truck and then re-entered, within a certain distance from the driver’s seat, the exosuit would automatically turn off so the user could sit down comfortably with the exosuit in disengaged mode. The reverse scenario could also be used to automatically engage the exosuit upon exiting the truck and moving more than 5 feet from the driver’s seat (or other part of the truck), for instance. While in the truck, the exosuit battery could also be charged wirelessly (while the exosuit is worn by the user), for instance, by having a charging mat built into or under the vehicle’s seat, which might for instance, check the battery power of the exosuit. If the battery level were below a specified threshold then wireless recharging of the battery would occur while the user was seated in the vehicle. As such, power and/or control signals could be transmitted wirelessly to the exosuit.

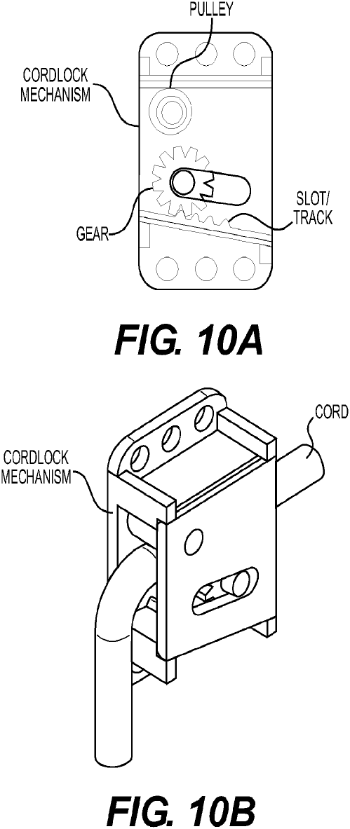

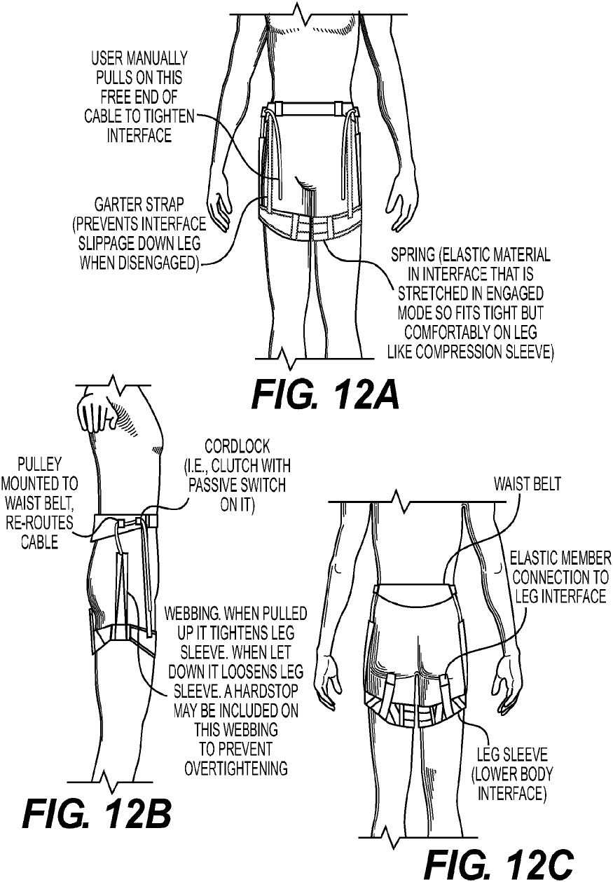

FIGS. 10A-10B are drawings illustrating a cordlock mechanism which can be part of an engagement/disengagement system of an exosuit. The cordlock mechanism (or cordlock) may be located on a waist belt (such as shown in FIGS. 12A-15C), or alternatively on the interface or another part of the exosuit. FIGS. 10A-10B show an example of a cordlock mechanism (a type of unidirectional clutch) in which a cable (cord) passes between a gear and pulley which are oriented to allow the cable to slide easily in one direction (toward the right in FIGS. 10A-10B) but not in the other direction. An example of how this could be used in exosuit is that a user could manually pull the cable to the right to engage the elastic members and/or tighten an interface on the body. Alternatively, a powered actuator (e.g., containing a motor) could provide this pulling function. A switch on this cordlock mechanism could then move the gear to the right and down in the slot so that it releases (un-pinches) the cable and allows it to slide freely back to the left (e.g., with the pulling force coming from a retractable; spring). This would then allow the elastic members to disengage and/or for a body interface to loosen. FIG. 10A is a 2D, partially transparent view. FIG. 10B is a 3D view. The gray cylinder is the cable, the gear and pulley are shown in gold in the 2D view.

How the cordlock mechanism locks and unlocks: When the cord is pulled to the right, the gear moves to the right and down, allowing the cord to freely move and slide through. When the cord is pulled to the left, it drags the gear back up the track/slot (due to friction between the cord and gear), which then pinches the cord and locks it in place, preventing it from sliding further to the left. To unlock the cordlock mechanism, the user pulls the cording forward slightly to shift the gear forward and down in the slot, and then lets the cording freely slide to the left while holding the cord at an upwards angle (to prevent friction from pulling the gear back up the track). In effect, the gear moving up or down the slot provides the switch function. Alternatively the gear can be biased towards to the left by a spring, to ensure the unidirectional locking/pinching occurs, and then an additional switch would be toggled by the user or by a motor to move the gear down the slot when cord needed to slide freely to the left.

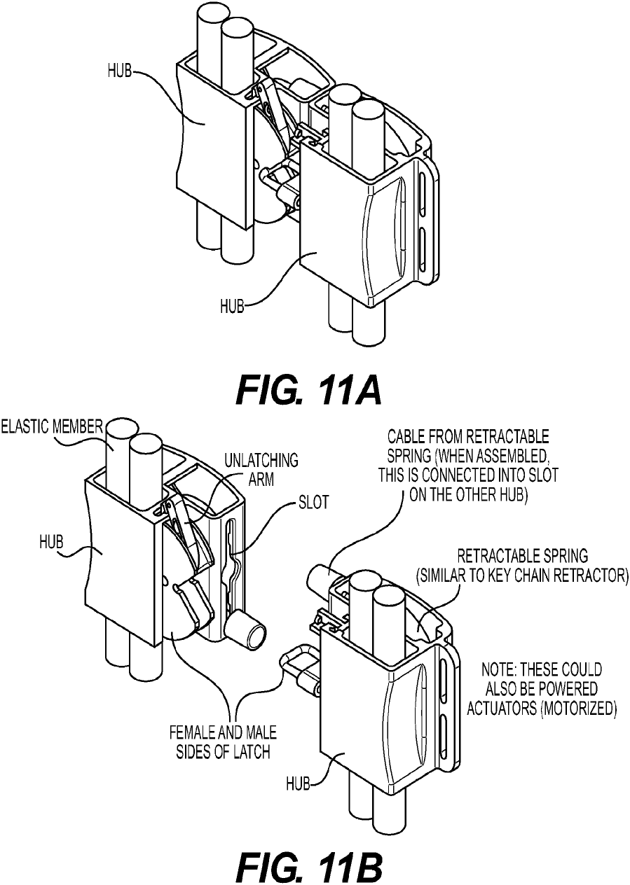

FIGS. 11A-11B are drawings illustrating hubs which are a part of an engagement/disengagement system of an exosuit. The figures show one embodiment of hubs with a built-in retractable spring and an automatic latching/unlatching function. FIG. 11A shows engaged mode and FIG. 11B shows disengaged mode. The retractable springs bias the system towards engaged mode, and pull the hubs together with enough force for the latch to lock into place in engaged mode. To disengage: one or more cables (not depicted) would attach to the hub and also to the unlatching arm; when that cable was pulled (e.g., manually, by spring, by motor) then it would unlatch the hubs and also pull the hubs to the side of the wearer. That locking/unlocking could also be implemented in many other ways, such as using magnets, motors or other locking/unlocking mechanisms.

FIGS. 12A-12C are drawings illustrating loosening/tightening of a lower body interface (of an exosuit worn by a user) comprising a (e.g., leg) sleeve, in an engaged (tight / form-fitting interface) mode. This embodiment is passive (unpowered, manual).

How engaged mode functions: The leg sleeve has a spring (elastic material) built-in. When the sleeve is tightened in engaged mode, the spring is stretched. When the leg sleeve is switched to disengaged mode, the spring in the leg sleeve helps or causes the sleeve to loosen. There is a webbing loop on the outer side of the leg sleeve that is attached to cording that is routed through a pulley and cordlock (or cordlock mechanism) located on the belt. The cordlock shown in FIGS. 12A-15C can be the type of cordlock mechanism shown in FIGS. 10A-10B described more fully above. When switching to engaged mode, the cording is pulled forward through the belt and locked into place with the cordlock. When that cording is pulled, it pulls on that webbing loop on the outer side of the leg, and tightens the leg sleeve.

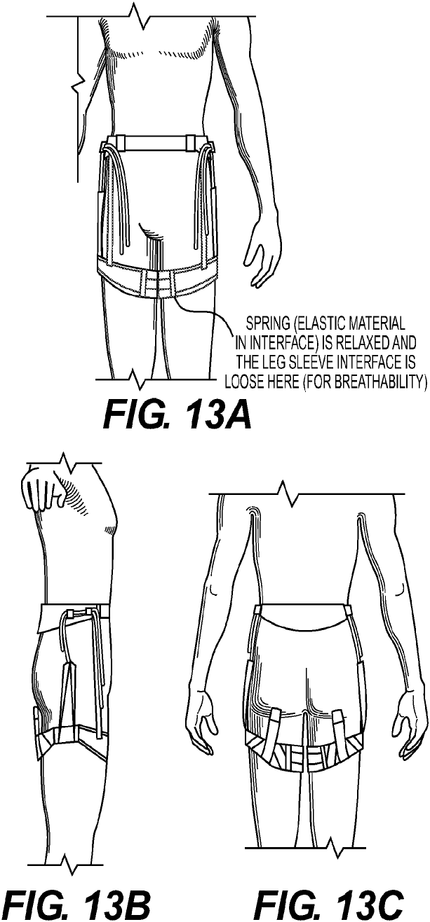

FIGS. 13A-13C are drawings illustrating loosening/tightening of the leg sleeve shown in FIGS. 12A-12C, in a disengaged (loose interface) mode.

How disengaged mode functions: When the cord is released by the cordlock then the tension in the webbing and leg sleeve is also reduced. The spring in the leg sleeve retracts back to its nominal length, pulling the webbing on the outer side of the leg downward and creating more slack circumferentially in the leg sleeve.

Alternatively, the embodiment of FIGS. 12A-13C may be powered (motorized). A powered actuator unit may be employed and may include an actuator (e.g., motor) and power supply. The powered actuator may also contain a processor, sensors, telemetry, switch, etc. The powered actuator provides the pulling force on the green cable to tighten, and removes force (spools out cable) to loosen the interface. The powered actuator could be controlled many different ways (e.g., control algorithm on processor, remote signals, user input). How this functions: Instead of a manual cordlock, the leg sleeves rely on a motor/actuator to loosen and tighten. The routing of the leg sleeve is the same, except that the cording is wrapped around a spool controlled by the motor. The modes are changed by flipping a switch or a controller that changes the direction that the spool spins. The powered actuator could be located on other parts of the user’s body or exosuit, including on the interface itself.

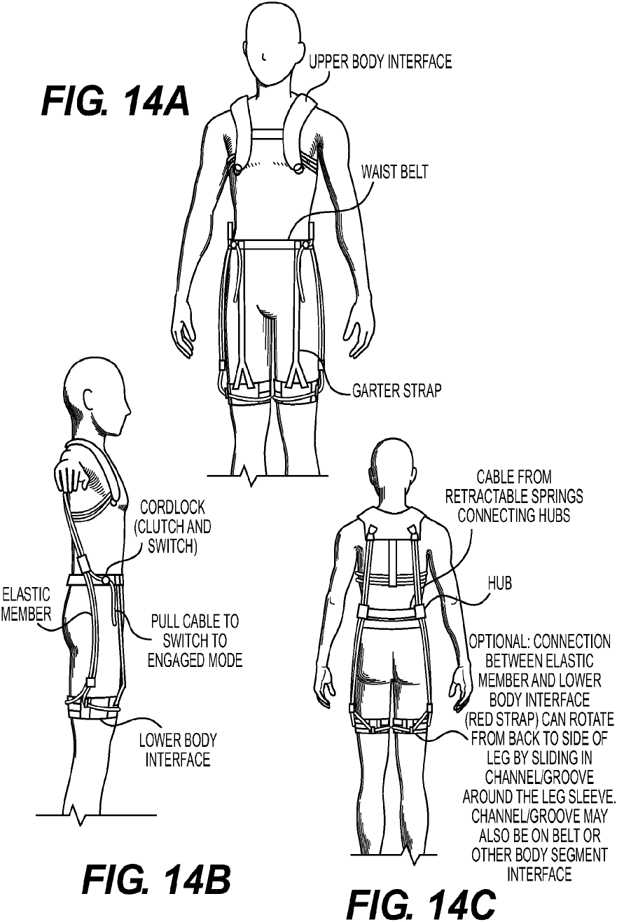

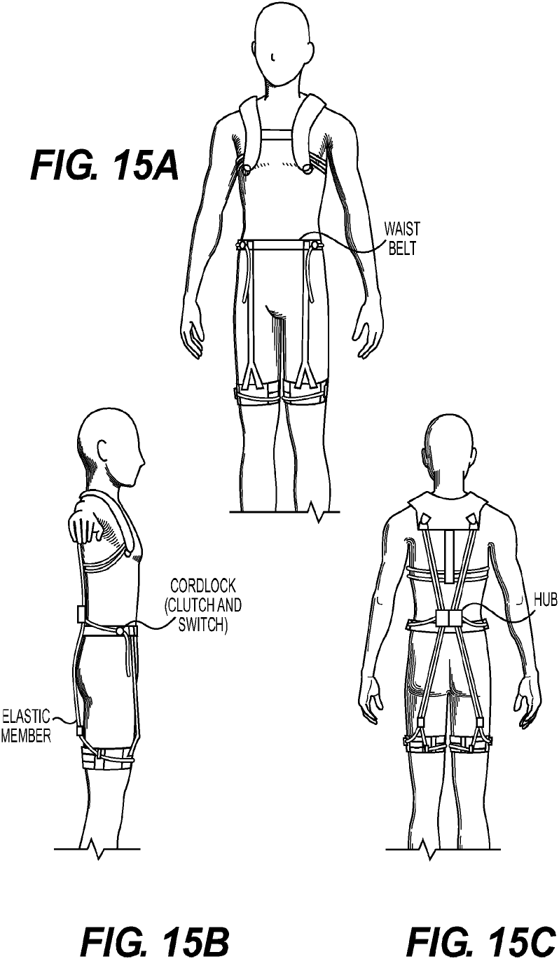

FIGS. 14A-14C are drawings illustrating an exosuit worn by a user (and which is similar to the embodiment in FIGS. 13A-13C), in a disengaged mode, but the leg sleeves do not tighten/loosen in this embodiment. FIGS. 15A-15C are drawings illustrating the exosuit shown in FIGS. 14A-14C, in an engaged mode. In this embodiment, the leg sleeves do not loosen and tighten with mode switches. The process for switching to disengaged mode has manual pull cording and is locked in place with a cord lock, and the process for switching to engaged mode happens automatically with retractable springs integrated into the hubs (such as hubs in FIGS. 11A-11B).

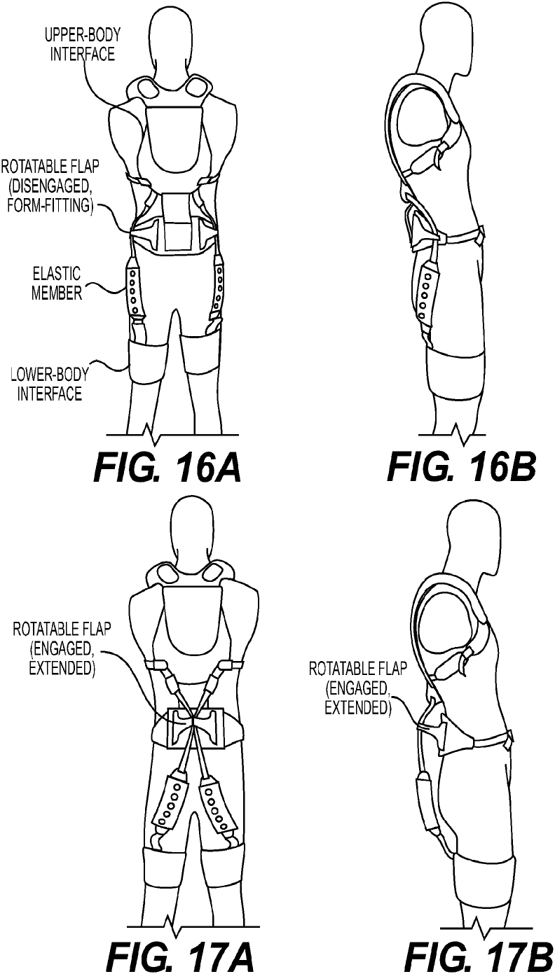

FIGS. 16A-16B are drawings illustrating an exosuit worn by a user and having an engagement/disengagement system comprising extensible moment arms (flaps) which are non-extended, in a disengaged mode. FIGS. 17A-17B are drawings illustrating the engagement/disengagement system shown in FIGS. 16A-16B comprising extensible moment arms (flaps) which are extended, in an engaged mode. The mode-switching here uses a rotatable flap and/or hinge design. Alternatively, the hinges could be spring-loaded (via a biasing spring), or designed as a bi-stable mechanism, such that the exosuit will stay in its current state (engaged or disengaged) even without the need for a clutch. In use, the bimodal exosuit shown in FIGS. 16A-17B may be disengaged when a user of the exosuit is seated and walking/carrying, and may be engaged when a user of the exosuit is bending or lifting.

FIGS. 22A-22B are drawings illustrating loosening/tightening of an upper body interface (of an exosuit worn by a user) comprising, for example, a vest, in a disengaged (loose interface) mode. FIGS. 23A-23B are drawings illustrating loosening/tightening of the vest shown in FIGS. 22A-22B, in an engaged (tight / form-fitting interface) mode. The vest has lacing (cording) in the front, connected to the elastic members. The vest tightens as elastic members get pulled to the back in engaged mode and loosens as elastic members get pulled to the side for disengaged mode. The vest may also contain or attach to additional passive (e.g., spring) or powered (e.g., motorized) elements that cause it to automatically loosen or pop open when switched into disengaged mode.

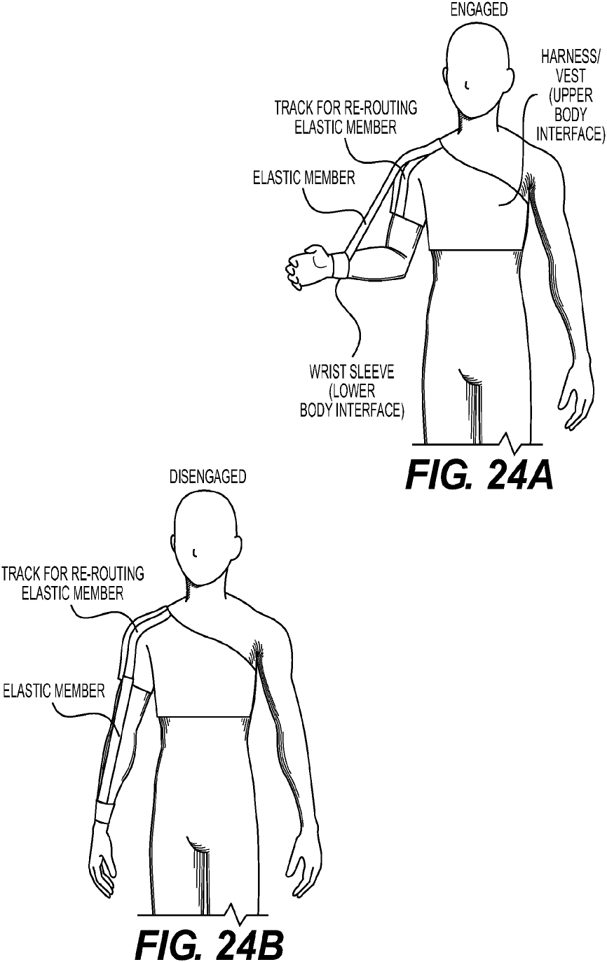

FIG. 24A is a drawing illustrating an exosuit (i.e., an arm exosuit) worn by a user and assisting a body segment other than the back, in an engaged mode. FIG. 24B is a drawing illustrating the arm exosuit shown in FIG. 24A, in a disengaged mode. This embodiment includes an arm exosuit with an elastic member traversing an upper arm of a user. A track is employed for the elastic member to traverse along the shoulder and bicep. When in engaged mode, an end of the elastic member sits at the top of the shoulder and the opposite end of the elastic member is connected to the wrist. That keeps the elastic member taut and makes it easier for the arm to be held up in the air for an extended period of time. In disengaged mode, the elastic member end that was at the top of the shoulder slides down the track, from shoulder to bicep, to give the arm enough slack to move freely. The interfaces could also be built to loosen and tighten for comfort. Alternatively, the track could be oriented to slide laterally to the side, to route the elastic member along the side of the arm.

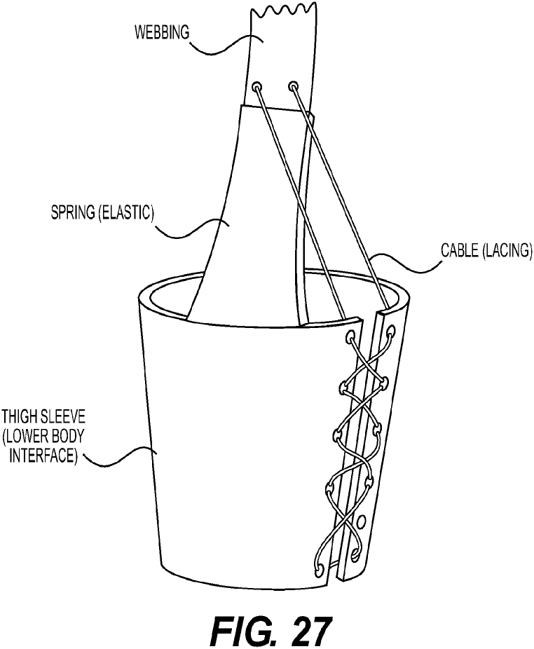

FIG. 27 is a drawing illustrating a perspective view of a spring (elastic element) attached to webbing (or other elastic member) and a thigh sleeve. An elastic cable is laced through the thigh sleeve and is attached to the (same) webbing. When the user enters into a squat or a lean, that piece of webbing pulls up and tensions the spring while simultaneously pulling the cable that is laced through the thigh sleeve, causing it to tighten. When the user is no longer squatting or leaning, the tension in the spring is reduced, allowing it to serve as a return spring so that the lacing in the thigh sleeve now has the slack necessary to loosen.

FIG. 19 is a drawing illustrating loosening/tightening of a lower body interface (of an exosuit worn by a user) comprising a (e.g., leg) sleeve where a portion of the leg sleeve remains tight. In this embodiment the leg sleeve has an elastic band at the top of the leg sleeve that always remains tight (like compression shorts) to keep the leg sleeve in the same spot on the thigh. A panel with cording is positioned in the center front of the sleeve that can be loosened and tightened for comfort. More specifically, a small portion of the thigh interface (or any of the interfaces on other body segments described in this disclosure) may always remain tight in order to prevent migration/movement of the interface when loose/disengaged. Part or all of the (e.g., thigh) interface may loosen or tighten. For instance, in one embodiment a thin strip of elastic material would comprise the top-most portion of the thigh sleeve, such as to maintain light compression on the leg at all times (similar to spandex or the elastic waist band in boxer briefs). In this embodiment, there would be no need for a garter belt or other connection from the waist down to the thigh sleeve to prevent the thigh sleeve from sliding down the leg when it was loose in disengaged mode. Rather this top-most elastic portion of the thigh sleeve would support the weight of the sleeve and hold it in place while in disengaged mode. Then in engaged mode a part of the thigh sleeve (e.g., the remainder of the thigh sleeve beyond the top-most elastic portion) would tighten down to provide more surface area over which to apply force. Alternatively, an extra cable, strap, or connection (e.g., from a waist or hip belt to the thigh sleeve may be employed to prevent the thigh interface from migrating or falling/slipping off the body segment (thigh) or from moving down the thigh.

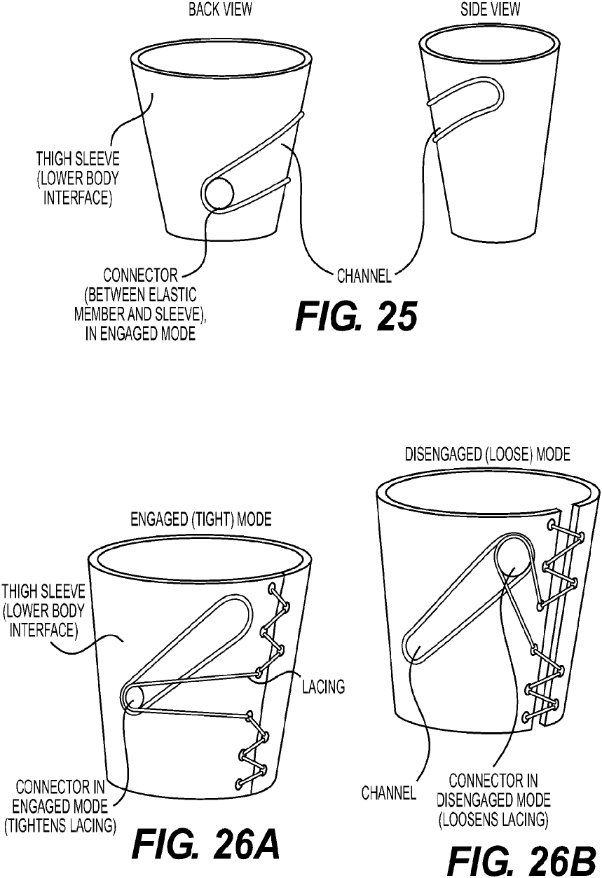

FIG. 25 is a drawing illustrating a back view (in an engaged mode) and a side view (where the connector would be located in the upper right portion of the channel, in a disengaged mode) of a thigh interface comprising a channel/track/groove configured to allow a connector to the elastic member (or attachment point as shown in the figure) to slide therein, wherein the connector is coupled to a spring (or other elastic member). The channel may be formed, for example, using a ratchet track like that used in some belts, a piping track with a slider like that used on some backpack straps with a sternum slider, or the way a zipper head moves up and down a zipper. The channel could be formed from low-friction plastic (or similar materials), such that another low-friction plastic component (i.e., a connector) could slide to effect engaged and disengaged modes. The channel’s function is to allow the elastic member (or connector) to have a path to move back and forth across the thigh sleeve in order to position the elastic member at the center back of the thigh in an engaged mode, and at the outer side of the thigh in disengaged mode. That can be used for thigh sleeves that do not loosen and tighten when switching between modes. Alternatively, for sleeves intended to loosen and tighten, the tightening mechanism can be integrated into that channel/track in such a way that sliding into the engaged mode also engages the thigh sleeve and tightens it, and sliding into the disengaged mode disengages the thigh sleeve and loosens it.

FIGS. 26A-26B are drawings illustrating perspective views of a thigh sleeve interface (in engaged (tight) mode and disengaged (loose) mode, respectively) comprising a channel (or groove) in an outer layer thereof, similar to the channel (or groove) shown in FIG. 25. Cables attached to a slider/connector on the track are laced in the sleeve similar to corset lacing. The slider tightens or loosens the lacing, depending on the direction that it moves. FIGS. 26A-26B show a possible exemplary iteration of the track triggering engagement/disengagement of the leg sleeves. In this configuration, when the spring connector on the thigh sleeve slides into the disengaged mode, it should: 1) slacken the assistive elastic member; 2) re-route the elastic member to, for example, run over the sides or front of the body of the user (or to a different route from when in the engaged mode); and/or 3) loosen the thigh sleeves (using a dynamic loosening mechanism similar to shoe laces or other adjustable tension lacing systems). Note items 1) - 3) could all occur by moving the spring connector along the thigh sleeve channel.

There may also optionally be a mechanism to prevent the loosened thigh sleeves from sliding down the thigh. Examples would be: 1) a strap running from a waist or hip belt to a thigh sleeve; or 2) a magnet on the belt that attracts a magnet on the elastic member. In another example, garter belts attached to a waist belt may be employed to prevent the thigh sleeves from sliding down legs when loosened.

A wearable assistance device may have more than two interfaces, and could also have more than two modes (e.g., disengaged with tension level one vs. engaged with tension level two vs. engaged with tension level three). See also the embodiments described above with respect to FIGS. 21A-21C. There could also be an infinite number of modes in the case of exosuits that include mechanisms that allow for continuous/infinite adjustment of elastic member stiffness or tension (e.g., via direct motor control), or that allow for continuous/infinite adjustment of interface tightness or looseness.

Embodiments described herein are directed to a wearable assistance device to be worn, at least partly, on a back of a user, but the same or similar type of wearable assistance device could alternatively be applied to assist other body segments such as the arms, ankles, knees, hips, elbows, wrists or neck. The upper body and lower body interfaces do not necessarily have to be located on the trunk and the thigh of a user. Instead, the upper body and lower body interfaces could both be placed on a single body part, e.g., on bicep (or shoulder) and forearm (or wrist) portions of a single arm, with the engagement/disengagement system between them (see, for example, the exosuit shown in FIGS. 24A-24B described above). In this configuration, the upper body interface would be the bicep (or shoulder) interface, and the lower body interface would be the forearm (or wrist) interface. Instead of an exosuit, embodiments of the wearable assistance device and/or engagement/disengagement system could be integrated into clothing items or wearable accessories, such as a baby carrier, bra, backpack, or body armor.

Embodiments are directed to a wearable assistance device configured to be worn by a user. The device includes: an upper body interface; a lower body interface; one or more elastic members, each of the elastic members mechanically coupling the upper body interface to the lower body interface, and extending from the upper body interface to the lower body interface along a first route traversing a body segment of the user, to form an engaged mode, to apply an assistive force to and/or assistive moment about the body segment of the user; and an engagement/disengagement system mechanically connected to the one or more elastic members that allows the one or more elastic members to move, shift or rotate from along the first route traversing the body segment in the engaged mode to a second route different than the first route, to form a disengaged mode, that slackens the one or more elastic members and/or lessens or prevents the assistive force applied to the body segment.

In an embodiment, the lessening of assistive force in disengaged mode may only be applicable for certain (but not all) body postures. For instance, when standing vertically, the force in an elastic member may be zero (or close to zero) for both engaged and disengaged modes. But during a bent forward posture, the force in the elastic member will be higher in engaged mode, and lower in disengaged mode, due to body geometry effects, or differences in slack length of the elastic member in engaged versus disengaged mode, or due to force applied or controlled by an optional actuator, or combinations thereof.

In an embodiment, the upper body interface or lower body interface is worn on the upper-body or lower-body, respectively, of the user via a first tension when in the engaged mode, wherein the first tension is decreased to a second tension when in the disengaged mode.

In an embodiment, the upper body interface is worn on a upper torso or shoulders of the user, and the lower body interface is worn on a thigh of the user.

In an embodiment, the body segment of the user is a back of a torso of the user, wherein the second route traverses a side of the torso of the user.

In an embodiment, the engagement/disengagement system comprises at least one resecurable fastening device that locks the engagement/disengagement system in the engaged mode or the disengaged mode. The at least one resecurable fastening device may be selected from the group consisting of strap, string, cable, hook-and-loop fastener, clip, hook, zipper, magnet, snap, button, latch, clamp, buckle, and combinations thereof.

In an embodiment, the fastening device mechanically couples a first of the elastic members to a second of the elastic members such that there is no separation between the first and second of the elastic members when in the engaged mode, and the fastening device allows separation between the first and second of the elastic members when in the disengaged mode.

In an embodiment, the fastening device mechanically couples a first of the elastic members to a second of the elastic members at a first distance apart when in the engaged mode, and at a second distance apart larger than the first distance apart when in the disengaged mode. Alternatively, the fastening device mechanically couples a first of the elastic members to a second of the elastic members at a first distance apart when in the disengaged mode, and at a second distance apart larger than the first distance apart when in the engaged mode.

In an embodiment, the lower body interface is worn on a body part of the user via a first tension when in the engaged mode, wherein the first tension is decreased to a second tension when in the disengaged mode.

In an embodiment, the body part is a thigh of the user, wherein the lower body interface comprises a channel having a first end and a second end opposite the first end, wherein the channel traverses a portion of the lower body interface, and wherein one of the elastic members is mechanically coupled to the lower body interface via a connector configured to slide within the channel such that the connector is closer to the first end of the channel when in the engaged mode, and is closer to the second end of the channel when in the disengaged mode.

In an embodiment, the further comprises a belt positioned on or above a waist or hips of the user, wherein the lower body interface is worn on a thigh of the user via a first tension when in the engaged mode, wherein the first tension is decreased to a second tension when in the disengaged mode, and wherein the belt is mechanically coupled to the lower body interface to prevent the lower body interface from moving down the thigh when in the disengaged mode.

In an embodiment, the engagement/disengagement system comprises cording configured to pull the elastic members toward each other when moving from the disengaged mode to the engaged mode.

In an embodiment, the engagement/disengagement system comprises hinges or axes configured to pull the elastic members toward each other when moving from the disengaged mode to the engaged mode.

In an embodiment, the engagement/disengagement system comprises slidable hubs that are affixed to each other and are slidable along the elastic members, wherein the slidable hubs are configured to be positioned at upper portions of the elastic members in the disengaged mode, and the slidable hubs are configured to be lowered to positions substantially mid-way between ends of the elastic members such that the elastic members are brought together in the engaged mode.

In an embodiment, the engagement/disengagement system comprises flaps and hinges or axes, wherein the flaps are configured to rotate about the hinges or axes whereby the elastic members are pulled toward each other into the engaged mode, thereby resulting in an extended moment arm of the elastic members about the body segment.

Embodiments are also directed to a wearable assistance device configured to be worn by a user. The device includes an interface and an elastic member mechanically coupled to the interface via a connection system via a first force when in an engaged mode, and the elastic member is mechanically coupled to the interface via the connection system via a second force less than the first force when in a disengaged mode. The interface is configured to be worn on a body part of the user via a first tension when in the engaged mode, and via a second tension less than the first tension when in the disengaged mode. Higher tension in the interface creates more compression around the body segment under the interface. And less tension in the interface results in less compression of the body segment under the interface.

In an embodiment, the connection system comprises a connector, wherein the interface comprises a channel having a first end and a second end opposite the first end, wherein the channel traverses a portion of the interface, and wherein the connector is configured to slide within the channel such that the connector is closer to the first end of the channel when in the engaged mode, and is closer to the second end of the channel when in the disengaged mode.

In an embodiment, the interface comprises a sleeve and the body part is a thigh, leg, torso, pelvis, forearm, or upper arm of the user.

In an embodiment, the device further comprises an actuator, wherein the interface comprises a sleeve, and wherein the engaged mode and/or disengaged mode is activated via the actuator.

In an embodiment, the actuator comprises a mechanism selected from the group consisting of a motor, gear, pneumatic actuator, hydraulic actuator, magnetic actuator, solenoid, spring, power source, and combinations thereof.

In an embodiment, there is another actuator that applies or modulates tension force along, or perpendicular to, the elastic member. For instance, the actuator may slacken one or more the elastic members in disengaged mode, or may perform mechanical work on one or more elastic members as a user moves in engaged mode. The actuator comprises a mechanism selected from the group consisting of a motor, gear, pneumatic actuator, hydraulic actuator, magnetic actuator, solenoid, spring, power source, and combinations thereof. Actuators may also contain other components such as sensors, processors, or mechanical transmissions.

In an embodiment, the interface comprises a sleeve and the elastic member comprises webbing, wherein the connection system comprises: a spring in series with the webbing which is stiffer than the spring, wherein the spring is connected between the webbing and the sleeve; and a cable laced through the sleeve and attached to the webbing. The webbing is configured to pull up and increase tension to the spring while simultaneously pulling the cable that is laced through the sleeve, when the user enters into a squat or a lean, causing the sleeve to tighten, and wherein the tension in the spring is configured to be reduced, allowing the spring to serve as a return spring, when the user is no longer squatting or leaning, so that the cable laced through the sleeve has slack necessary to loosen the sleeve.

In any of the embodiments above, there could be a powered actuator unit (including, for example, motor, battery, processor, sensor, and wireless transmitter/receiver) on each interface that receives a signal and then loosens or tightens the interface. Or there could be a powered actuator unit worn on the belt that connects to the cording, and transmits a pulling force to replace the need for the user to manually switch modes. That actuator unit would serve the same function to re-route the elastic members to be in engaged or disengaged mode. Those actuator units would take an input, from the user and/or sensors, and could be a simple on/off control, or controlled by automated algorithms on the processor or elsewhere (e.g., using Internet-of Things (IoT), or a nearby computer/controller).

In any of the embodiments above, engaged/disengaged mode switching could be controlled by inertial measurement units (sensors) that track motion, or muscle activity (e.g., electromyography) sensors, or pressure insole sensors, or various other sensors that track motion or biometric data, or sensors that integrate signals from off-the-person, such as proximity sensors.

In any of the embodiments above, the switch or powered actuator could be placed anywhere on the exosuit or anywhere on the user’s body.

In any of the embodiments above, the mode-switching can be powered (motorized) or passive.

In any of the embodiments above, the exosuit may have a clutch, a clutch switch coupled to the clutch, a sensor coupled to the clutch switch, a transmitter coupled to the sensor, or an interface motor coupled to a receiver that receives a (wired or wireless) signal from the sensor that actuates the switch to tighten or loosen the thigh interface. The sensor and transmitter may be contained within a single transceiver.

In any of the embodiments above, when switched from disengaged mode to engaged mode, the thigh sleeve could tighten down to provide a stronger connection/anchor to the leg, to support assistive forces from the elastic member. The tightening could be achieved by various mechanisms such as pulling on a strap, cable, or cord that tightens lacing (similar to a corset or shoe strings) around the body part (e.g., thigh), or zipping a zipper that cinches the interface around the body part as it closes, or using pneumatics to adjust air pressure within an inflatable interface to effectively tighten or loosen the interface. There may optionally be one or more motors employed to control the tightening or loosening of the interfaces (e.g., thigh sleeves), for instance, by attaching a motor or other actuator to control the tensioning of the lacing system. In one embodiment, that could be achieved by attaching a motor and gear system to a spool, such that rotating the motor in one direction would wind a cable (which is connected to or part of the lacing) around the spool causing the interface to tighten, and then rotating the motor in the opposite direction would release the cable from the spool allowing the interface to loosen on the body part. A sensor may control the tightening or loosening (e.g., a sensor that monitors the optional clutch as being on or off) or other control inputs. Control inputs may come directly from the user (e.g., voice control, button press), or may result from an automated algorithm that uses muscle activity, motion sensing and/or other biometric data to activate the motor to determine when to tighten and loosen the interface. The thigh sleeves can loosen in disengaged mode (for comfort, to feel more like loose shorts) and then tighten around the leg during engaged mode to transmit assistive forces to the legs. Any of these thigh sleeve embodiments could be used with any of the bimodal wearable assistance devices described above, or within other wearable assistance devices, or for interfacing with other body parts.

In any of the embodiments above, the loosening/tightening of any interface can be used with and triggered at the same time as (or slightly before or after) the disengagement/engagement of the elastic members.

In any of the embodiments above, the loosening/tightening of the thigh/leg sleeves could be used alone or with other exoskeleton or exosuit designs.

Any benefit/concept/application in the use of the thigh/leg sleeves (such as loosening/tightening) in any of the above embodiments may be applicable to sleeves employed with any other body segment.

In any of the embodiments above, loosened body interfaces can improve thermal comfort and reduce skin temperature relative to tight, form-fitting interfaces. The inventor has found that the skin under the loose sleeve was 4-6° F. cooler after 25 minutes of physical activity, and two of four participants reported the loose sleeve improved their thermal comfort. After completion of the physical activity, the form-fitting sleeve was loosened, causing a 2-9° F. drop in skin temperature underneath for all participants, and causing two participants to report improved thermal comfort. These preliminary findings confirmed that an exosuit that can quickly loosen its interface when assistance is not required (and re-tighten when assistance is required) has the potential to enhance thermal comfort for some individuals and environments.

Although embodiments are described above with reference to an exosuit, the exosuit described in any of the above embodiments may alternatively be another type of wearable assistance device such as an exoskeleton. Such alternatives are considered to be within the spirit and scope of the present invention, and may therefore utilize the advantages of the configurations and embodiments described above.

In addition, although embodiments are described above with reference to an exosuit with particular loosening/tightening functionality, the exosuit described in any of the above embodiments may alternatively have different ways to achieve the same loosening/tightening functionality, and/or may alternatively have different type(s) of loosening and/or tightening functionalities. For example, in some embodiments, there may be a reset spring that will bias an interface to nominally be either loose or tight. Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this application may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A wearable assistance device configured to be worn by a user, the device comprising:
an upper body interface;
a lower body interface;
one or more elastic members, each of the elastic members mechanically coupling the upper body interface to the lower body interface, and extending from the upper body interface to the lower body interface along a first route traversing a body segment of the user, to form an engaged mode, to apply an assistive force to and/or assistive moment about the body segment of the user; and
an engagement/disengagement system mechanically connected to the one or more elastic members that allows the one or more elastic members to move, shift or rotate from along the first route traversing the body segment in the engaged mode to a second route different than the first route, to form a disengaged mode, that slackens the one or more elastic members and/or lessens or prevents the assistive force applied to the body segment.

2. The device of claim 1, wherein the upper body interface or lower body interface is worn on the upper-body or lower-body, respectively, of the user via a first tension when in the engaged mode, and wherein the first tension is decreased to a second tension when in the disengaged mode.

3. The device of claim 1, wherein the upper body interface is worn on a upper torso or shoulders of the user, and the lower body interface is worn on a thigh of the user.

4. The device of claim 3, wherein the body segment of the user is a back of a torso of the user, and wherein the second route traverses a side of the torso of the user.

5. The device of claim 1, wherein the engagement/disengagement system comprises at least one resecurable fastening device that locks the engagement/disengagement system in the engaged mode or the disengaged mode.

6. The device of claim 5, wherein the fastening device mechanically couples a first of the elastic members to a second of the elastic members such that there is no separation between the first and second of the elastic members when in the engaged mode, and the fastening device allows separation between the first and second of the elastic members when in the disengaged mode.

7. The device of claim 5, wherein the fastening device mechanically couples a first of the elastic members to a second of the elastic members at a first distance apart when in the engaged mode, and at a second distance apart larger than the first distance apart when in the disengaged mode.

8. The device of claim 1, wherein the lower body interface is worn on a thigh of the user via a first tension when in the engaged mode, wherein the first tension is decreased to a second tension when in the disengaged mode, wherein the lower body interface comprises a channel having a first end and a second end opposite the first end, wherein the channel traverses a portion of the lower body interface, and wherein one of the elastic members is mechanically coupled to the lower body interface via a connector configured to slide within the channel such that the connector is closer to the first end of the channel when in the engaged mode, and is closer to the second end of the channel when in the disengaged mode.

9. The device of claim 1, further comprising a belt positioned on or above a waist or hips of the user, wherein the lower body interface is worn on a thigh of the user via a first tension when in the engaged mode, wherein the first tension is decreased to a second tension when in the disengaged mode, and wherein the belt is mechanically coupled to the lower body interface to prevent the lower body interface from moving down the thigh when in the disengaged mode.

10. The device of claim 1, wherein the engagement/disengagement system comprises cording configured to pull the elastic members toward each other when moving from the disengaged mode to the engaged mode.

11. The device of claim 1, wherein the engagement/disengagement system comprises hinges or axes configured to pull the elastic members toward each other when moving from the disengaged mode to the engaged mode.

12. The device of claim 1, wherein the engagement/disengagement system comprises slidable hubs that are affixed to each other and are slidable along the elastic members, and wherein the slidable hubs are configured to be positioned at upper portions of the elastic members in the disengaged mode, and the slidable hubs are configured to be lowered to positions substantially mid-way between ends of the elastic members such that the elastic members are brought together in the engaged mode.

13. The device of claim 1, wherein the engagement/disengagement system comprises flaps and hinges or axes, and wherein the flaps are configured to rotate about the hinges or axes whereby the elastic members are pulled toward each other into the engaged mode, thereby resulting in an extended moment arm of the elastic members about the body segment.

14. A wearable assistance device configured to be worn by a user, the device comprising:
an interface; and
an elastic member mechanically coupled to the interface via a connection system via a first force when in an engaged mode, and the elastic member mechanically coupled to the interface via the connection system via a second force less than the first force when in a disengaged mode, wherein the interface is configured to be worn on a body part of the user via a first tension when in the engaged mode, and via a second tension less than the first tension when in the disengaged mode.

15. The device of claim 14, wherein the connection system comprises a connector, wherein the interface comprises a channel having a first end and a second end opposite the first end, wherein the channel traverses a portion of the interface, and wherein the connector is configured to slide within the channel such that the connector is closer to the first end of the channel when in the engaged mode, and is closer to the second end of the channel when in the disengaged mode.

16. The device of claim 15, wherein the interface comprises a sleeve and the body part is a thigh, leg, torso, forearm, or upper arm of the user.

17. The device of claim 14, further comprising an actuator, wherein the interface comprises a sleeve, and wherein the engaged mode and/or disengaged mode is activated via the actuator.

18. The device of claim 17, wherein the actuator comprises a mechanism selected from the group consisting of a motor, gear, pneumatic actuator, hydraulic actuator, magnetic actuator, solenoid, spring, power source, and combinations thereof.
